(19) United States
(12) Patent Application Publication
(10) Pub. No.: US 2022/0218599 A1
(43) Pub. Date: Jul. 14, 2022

(54) SELF-EMULSIFYING DRUG DELIVERY (SEDDS) FOR OPHTHALMIC DRUG DELIVERY

(71) Applicant: Allergan, Inc., Irvine, CA (US)

(72) Inventors: Yumna Shabaik, Anaheim, CA (US); Jim Jiao, Irvine, CA (US); Chetan Pujara, Irvine, CA (US)

(21) Appl. No.: 17/455,375

(22) Filed: Nov. 17, 2021

Related U.S. Application Data
(63) Continuation of application No. 15/554,983, filed on Aug. 31, 2017, now abandoned.
(60) Provisional application No. 62/128,798, filed on Mar. 5, 2015.

(51) Int. Cl. A61K 9/00; A61K 47/10; A61K 47/26; A61K 47/44; A61K 31/573; A61K 47/14; A61K 9/107; A61K 31/498; A61K 31/542; A61K 31/568; A61K 31/138

(52) U.S. Cl. CPC A61K 9/0048; A61K 47/10; A61K 47/26; A61K 47/44; A61K 31/573; A61K 9/107; A61K 9/1075; A61K 31/498; A61K 31/542; A61K 31/568; A61K 31/138; A61K 47/14

(57) ABSTRACT

Provided herein are topical ophthalmic preparations which comprise a non-aqueous, self-emulsifying system which can spontaneously give rise to either nanosized emulsions upon contact with an aqueous phase. Also provided herein are methods for the preparation of the same and their use in formulating and delivering poorly water soluble drugs.

### CROSS REFERENCE TO RELATED APPLICATIONS

This application is a continuation of U.S. patent application Ser. No. 15/554,983, filed Aug. 31, 2017, which is a national stage application under 371 U.S.C. § 371 of PCT Application PCT/US2016/020507 filed Mar. 2, 2016, which claims the benefit of U.S. Provisional Application No. 62/128,798 filed on Mar. 5, 2015, which are incorporated by reference herein in their entireties and serve as the basis of a priority and/or benefit claim for the present application.

### FIELD OF THE INVENTION

Provided herein are novel ophthalmic compositions capable of undergoing self-emulsification. These compositions spontaneously self-emulsify when in contact with an aqueous medium, including but not limited to, the aqueous medium of the tear film. The resulting emulsions are in the sub-micron to nanometer range with respect to droplet size.

### BACKGROUND OF THE INVENTION

Bioavailability of drugs delivered through topical ophthalmic administration is estimated to be about 5% of the applied dose. Physiological conditions at this target site present multiple challenges for drug delivery which include poor permeability across the corneal membrane and short residence time due to tear drainage. These and other factors limit the exposure of the ocular tissues to drug and result in the extremely low bioavailability observed.

Formulations for ocular treatment are described, for example, in US Patent Publication No. 2006/0182771 A1. Ophthalmic compositions for the administration of liposoluble active ingredients are described in WO 2011/154985 A1. The addition of viscosity enhancers or use of polymers with thermal, pH or ion-sensitive gelling properties have been used to increase ocular residence time. The use of viscosity enhancers is limited by the fact that viscosity should not interfere with ease of application from a dropper bottle and addition of polymers may be precluded for biocompatibility reasons.

Self-emulsifying drug delivery systems (SEDDS) are isotropic mixtures of oil, surfactant (with or without co-surfactant) and co-solvent which spontaneously emulsify when exposed to an aqueous medium with gentle agitation. SEDDS have most commonly been studied to improve bioavailability of poorly water soluble drugs via oral administration. The addition of a co-solvent is important to the formation of a self-emulsifying system as it significantly reduces the interfacial tension. In so doing, it creates a fluid interfacial film with sufficient flexibility to take up different curvatures required to form microemulsion over a wide range of compositions.

The composition of the pre-concentrate oil, surfactant and co-solvent determines the nature of the resultant emulsion following dispersion in the aqueous phase. Microemulsions arising from SMEDDS (self-microemulsifying drug delivery system) are thermodynamically stable while regular emulsions are kinetically stable. According to the lipid formulation classification system (LFCS), SMEDDS are characterized by a higher content of water-soluble components. These systems can achieve smaller-sized droplet dispersions and optical clarity, which is a desirable characteristic for improving currently existing ophthalmic emulsion formulations. SNEDDS (self-nanoemulsifying drug delivery system) and their resultant nanoemulsions share many of the advantageous characteristics of SMEDDS and microemulsions, but with the limitation of being only kinetically stable dispersions.

The following references are provided as background:
Phase transition water-in-oil microemulsions as ocular drug delivery systems: In vitro and in vivo evaluation, International Journal of Pharmaceutics, 328 (2007) 65-71
Oil in water microemulsions for ocular delivery: Evaluation of ocular irritation and precorneal retention, Journal of Controlled Release, 111 (2006) 145-152
Formulation of self-emulsifying drug delivery systems, Advanced Drug Delivery Reviews, 25 (1), pp. 47-58
New perspectives on lipid and surfactant based drug delivery systems for oral delivery of poorly soluble drugs, Journal of Pharmacy and Pharmacology, 62 (11), pp. 1622-1636
Potentials and challenges in self-nanoemulsifying drug delivery systems, 2012, Expert Opinion on Drug Delivery, 9 (10), pp. 1305-1317
Role of excipients in successful development of self-emulsifying/microemulsifying drug delivery system (SEDDS/SMEDDS), Drug Development and Industrial Pharmacy, 39 (1), pp. 1-19
Self-emulsifying drug delivery systems (SEDDS): Formulation development, characterization, and applications, Critical Reviews in Therapeutic Drug Carrier Systems, 26 (5), pp. 427-521
Spontaneous emulsification: Mechanisms, physicochemical aspects, modeling, and applications Journal of Dispersion Science and Technology, 23 (1-3), pp. 219-268

There is an unmet need for improved ocular drug delivery. Some have described self-emulsifying compositions for ophthalmic applications, but these are aqueous compositions in which an oil-in-water emulsion is already present, rather than a desired non-aqueous SEDDs which may be used, for example, for ocular drug delivery of aqueous-sensitive drugs. See U.S. Patent Publication No. 2004/0185068.

Ocular drug delivery in a non-aqueous SEDDS formulation has not previously been disclosed, and has the potential to provide several advantages. Surfactant/co-surfactant combinations can often have an enhancing effect on the permeation of the drug into ocular tissue. Improved bioavailability from SEDDS formulations can also arise from phase converting systems in which a change in water content may increase the viscosity leading to prolonged ocular retention time. Bioavailability can also be improved due to the drug being delivered in a solubilized state and as a consequence of potential direct uptake of nano-sized particles by ocular tissues. Other advantages of SEDDS formulations include enhanced stability of the active pharmaceutical ingredient (API) sensitive to heat or hydrolytic degradation because these systems are non-aqueous and do not require processing at elevated temperatures during manufacture.

While self-emulsifying systems are known in the field as a method of formulating and delivering poorly water soluble drugs, the use of the self-emulsifying, pre-concentrate (i.e. non-aqueous formulation) in the form of an eye drop, with the purpose of achieving rapid and spontaneous emulsification in the tear fluid is a novel application. Currently, no known marketed topical ophthalmic medications are formulated as SNEDDS or SMEDDS pre-concentrates.

### SUMMARY OF THE INVENTION

Non-aqueous formulations capable of self-emulsification and their method of use and preparation are described. The identified formulations are intended for use as ophthalmic drug delivery vehicles which are capable of self-emulsification in an aqueous medium simulating tear fluid. In some embodiments, the oil component of the Self-emulsifying drug delivery systems (SEDDS) formulations is composed of a single long chain or medium chain triglyceride or medium chain mono-/di-glyceride. In other embodiments, the oil component is a blend of more than one oil comprised of a mono-/di-glyceride blended with either a long chain triglyceride or a medium chain triglyceride.

In some embodiments, the oil component may be a natural oil such as castor oil or a synthetic oil such as Captex® 355 or Capmul® MCM. The Captex® oil component may also be a combination of these oils.

In some embodiments, the surfactant may be Cremophor® ELP, Cremophor® RH-40 or Polysorbate 80.

In some embodiments, the co-solvent may be PEG 400, PEG 300 or Propylene Glycol.

In some embodiments, the SEDDS formulations may be used in combination with a therapeutic drug that is used to treat ophthalmic conditions and can be delivered topically to the eye.

The compositions provided herein are easy to prepare with few manufacturing steps that are simple and straight forward to follow.

### BRIEF DESCRIPTION OF THE FIGURES

FIG. 1 shows an exemplary process for manufacturing the SEDDS provided herein.

FIG. 2 shows a pseudo-ternary phase diagram showing the nano/microemulsion regions of system consisting of Castor Oil, Cremophor® ELP and PEG 300.

FIG. 3 shows a pseudo-ternary phase diagram showing the nano/microemulsion regions of system consisting of Castor Oil, Capmul® MCM, Cremophor® RH-40 and Propylene Glycol.

FIG. 4 shows a pseudo-ternary phase diagram showing the nano/microemulsion regions of system consisting of Captex®355, PS80 and PEG 400.

FIG. 5 shows a pseudo-ternary phase diagram showing the nano/microemulsion regions of system consisting of Capmul® MCM, Cremophor® RH-40 and Propylene Glycol.

FIG. 6 shows a pseudo-ternary phase diagram showing the nano/microemulsion regions of system consisting of Capmul® MCM, Cremophor® ELP and Propylene Glycol.

FIG. 7 shows a pseudo-ternary phase diagram showing the nano/microemulsion regions of system consisting of Capmul® MCM, PS80 and PEG 400.

FIG. 8 shows a pseudo-ternary phase diagram showing the nano/microemulsion regions of system consisting of Castor Oil, Capmul® MCM, Cremophor® ELP and PEG 400.

FIG. 9 plots viscosity as a function of aqueous dilution for system consisting of Castor Oil, Capmul® MCM, Cremophor® ELP and PEG 400.

FIG. 10 shows a pseudo-ternary phase diagram showing the nano/microemulsion regions of system consisting of Captex®355, Capmul® MCM, Cremophor® ELP and PEG 400.

FIG. 11 plots viscosity as a function of aqueous dilution for system consisting of Captex®355, Capmul® MCM, Cremophor® ELP and PEG 400.

FIGS. 12A-F show the dilution compatibility of formulations F1 through F11 with simulated tear fluid (STF).

FIGS. 13A-D show the dilutability of drug loaded formulations F12 and F13.

### DETAILED DESCRIPTION OF THE INVENTION

Novel non-aqueous ophthalmic compositions comprising isotropic mixtures of oil, surfactant(s) and a co-solvent have been identified. These compositions are self-emulsifying and do not require high shear homogenization or other forms of high energy mechanical agitation to form oil-in-water dispersions. The resulting oil-in-water emulsions contain nano-sized droplets and appear optically clear or transparent.

The identified compositions are additionally capable of self-emulsifying in situ in the aqueous medium of the tear film when applied directly to the eye as a non-aqueous, pre-concentrate, SEDDS formulation. Furthermore, the identified formulations can be prepared easily in a few simple steps. All of the components, oil, surfactant and co-solvent are added together in the appropriate amounts and mixed until homogenously combined. A lipophilic, poorly water-soluble drug can then be added and stirred until completely dissolved.

The identified compositions are well suited as vehicles for the topical delivery of therapeutic drugs to the surface of the eye for treatment of various indications. Compatibility with simulated tear fluid was confirmed with all formulations to ensure that the composition of the tear fluid would not negatively impact the ability of the SEDDS formulations to spontaneously disperse. The simulated tear fluid used herein is composed of sodium chloride, calcium chloride, sodium phosphate dibasic, lysozyme, albumin, mucin and purified water, with pH adjusted to about 7.2.

As provided herein, a “non-aqueous” ophthalmic compositions or formulation is one which substantially no water is intentionally added as a component or ingredient of the composition. In some embodiments, a “non-aqueous” ophthalmic compositions or formulation is one which contains no more than 1% by weight water. In some embodiments, the non-aqueous ophthalmic compositions provided herein contain less than 0.5%, less than 0.25%, less than 0.1%, less than 0.05%, or less than 0.01% by weight water. It is understood that “less than” a certain percentage of water refers to from zero to the specified amount, within acceptable ranges of the detection of water by instrumentation known to those skilled in the art.

As provided herein, a “poorly water-soluble drug” refers to pharmacologically active agent which has low solubility in water. In the compositions provided herein, the route of administration is topical application or instillation to the eye. Therefore, as provided herein a “poorly water-soluble drug” refers to solubility poor enough to render topical ophthalmic delivery of the drug impracticable. Historically, the criteria provided by the United States Pharmacopea (USP 34, 5.30) have guided practitioners with respect to the solubility of oral drugs. A Biopharmaceutics Classification System (“BCS”) has also been developed to classify drugs based on solubility, permeability, and other parameters relevant to bioavailability. See Gordon L. Amidon et al., AAPS Journal, 2009, 11(4): 740-746. The BCS system, when adapted for topical ophthalmic applications, can be used to classify “poorly water-soluble drugs” useful in the topical ophthalmic compositions provided herein. The dissolution factor is adapted for a simulated tear fluid as described herein, and the permeability factor may be adapted to the particular conditions at the surface of the eye.

In general, a “poorly water-soluble drug” refers to any drug that administration of its therapeutic dose cannot be achieved via a simple topical ophthalmic solution within an acceptable pH range (pH of about 4.5-8.0) and that a solubilization means such as micellar system, co-solvent, complexation, emulsion, or other approaches needs to be applied to solubilize the drug.

In some embodiments, the poorly water-soluble drug is selected from the group consisting of antibiotics, antivirals, antifungals, 4-pregenen-11β-17-21-triol-3,20-dione derivatives, anesthetics, anti-inflammatory agents including steroidal and non-steroidal anti-inflammatories, anti-allergic agents, immunosuppressants, and hypertension lowering agents. Examples of suitable drugs include, but are not limited to, cyclosporine, prednisolone, loteprednol, dexamethasone, testosterone, declomethasone, rimexolone, fluorometholone, betaxolol, levobetaxolol, cephalosporin, amphotericin, fluconazole, tetracycline, brimonidine, brinzolamide, nepafenac, besifloxacin, natamycin, neomycin, and livocabastine.

In some embodiments, the poorly water-soluble drug is a 4-pregenen-11β-17-21-triol-3,20-dione derivative of Formula I, or an enantiomer, diastereoisomer, hydrate, solvate, tautomer or pharmaceutically acceptable salt thereof:

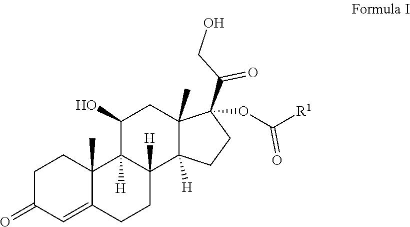

wherein:

R¹ is optionally substituted C₇-C₁₁ alkyl, optionally substituted C₂-C₈ alkenyl, optionally substituted C₂-C₈ alkynyl, optionally substituted C₄ or C₆₋₈cycloalkyl, optionally substituted aryl, substituted benzyl, optionally substituted heterocycle, optionally substituted C₃-C₁₀ cycloalkenyl, optionally substituted C₅-C₁₀ cyclodiene, optionally substituted (C₃-C₆) alkyl, amino groups, sulfonamide groups, amide groups, except phenyl.

These 4-pregenen-11β-17-21-triol-3,20-dione derivatives are described in U.S. Patent Publication No. 2013/0123223 (filed as Ser. No. 13/673,623), the entirety of which is hereby incorporated by reference. Additional examples within the scope of Formula I are provided below.

In some embodiments, the poorly water-soluble drug is a compound of Formula I above, wherein R¹ is

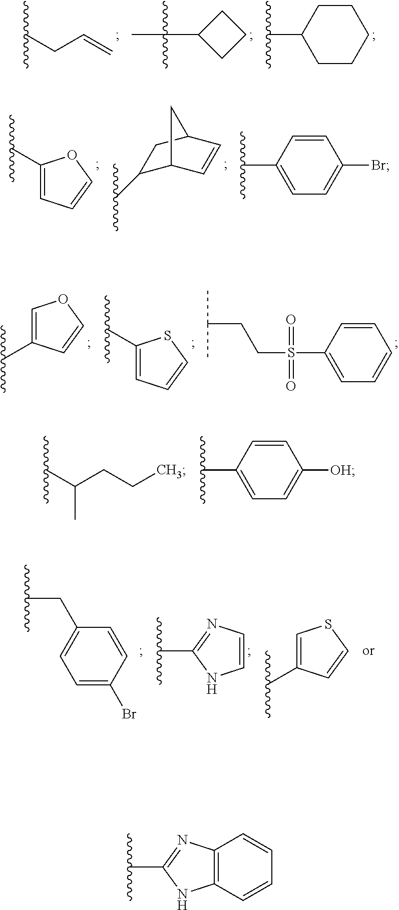

In some embodiments, the poorly water-soluble drug is a compound of Formula I, wherein R¹ is substituted aryl.

In some embodiments, the poorly water-soluble drug is a compound of Formula I, wherein R¹ is

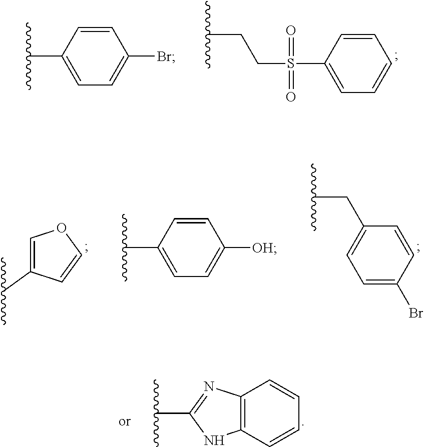

In some embodiments the compound of Formula I is:

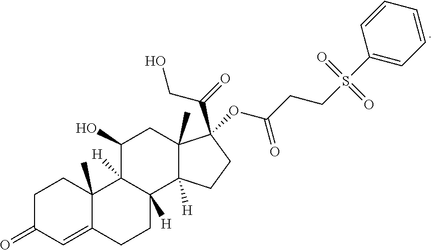 [SMILES: C[C@]12CCC(=O)C=C1CC[C@@H]1[C@@H]2[C@@H](O)C[C@@]2(C)[C@H]1CC[C@]2(OC(=O)CCS(=O)(=O)c1ccccc1)C(=O)CO]

In some embodiments the compound of Formula I is:

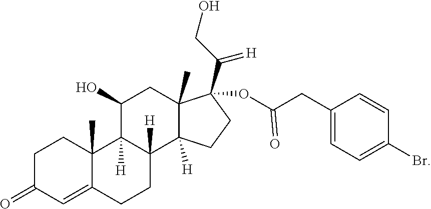

In some embodiments the compound of Formula I is:

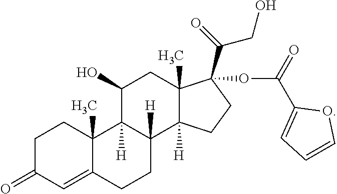

In some embodiments, the poorly water-soluble drug compound is one of the following 4-pregenen-11p-17-21-triol-3,20-dione derivatives, which are described in U.S. Patent Publication No. 2013/0123226 (filed as Ser. No. 13/673,074), the entirety of which is hereby incorporated by reference:
(8S,9S,10R,11S,13S,14S,17R)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl phenylacetate;
(8S,9S,10R,11S,13S,14S,17R)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl butyrate;
(8S,9S,10R,11S,13S,14S,17R)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl propionate;
(8S,9S,10R,11S,13S,14S,17R)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl octanoate;
(8S,9S,10R,11S,13S,14S,17R)-17-Glycoloyl-11-hydroxy-10,13-dimethyl-3-oxo-2,3,6,7,8,9,10,11,12,13,14,15,16,17-tetradecahydro-1H-cyclopenta[a]phenanthren-17-yl hexanoate;
(8R,9R,10S,11R,13R,14R,17S)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl benzoate;
(8S,9S,10R,11S,13S,14S,17R)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl heptanoate;
(8S,9S,10R,11S,13S,14S,17R)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl 2-methylpropanoate; and
(8R,9R,10S,11R,13R,14R,17S)-17-glycoloyl-11-hydroxy-10,13-dimethyl-3-oxo-2,3,6,7,8,9,10,11,12,13,14,15,16,17-tetradecahydro-1H-cyclopenta[a]phenanthren-17-yl rel-cyclopentanecarboxylate.

Conventional emulsions are thermodynamically unstable systems with relatively large droplet sizes, and which typically exhibit a milky appearance. While an emulsion is a dispersion of water or oil stabilized by only surfactant(s), emulsion droplets tend to coalesce over time, which may lead to phase separation.

By contrast, the SEDDS formulations provided herein form “nanosized emulsions” wherein such emulsions comprise a dispersion of a hydrophilic and a hydrophobic phase (e.g., oil and water) stabilized by a surfactant and optionally a co-surfactant, such dispersion characterized by containing nano-sized droplets. As provided herein, “nano-sized” droplets may have an average droplet size less than about 1000 nm, for example from about 5 to 800 nm, from about 10 to 600 nm, from about 10 to about 500 nm, from about 20 to about 200 nm, from about 10 to about 200 nm, and smaller ranges encompassed therein. Due to the nano-sized droplets, nanosized emulsions will usually be optically transparent.

As provided herein, nanosized emulsions may form microemulsions or nanoemulsions. A “microemulsion” is a dispersion of water or oil stabilized by using surfactant and co-surfactant to reduce interfacial tension, and is usually characterized by small droplet sizes (typically droplets less than 200 nm in diameter), thermodynamic stability, and a transparent appearance. A “nanoemulsion” refers to an emulsion with droplet sizes in nanometer range (typically less than 200 nm in diameter) and a transparent appearance, but which is thermodynamically unstable due to high interfacial tension at the oil and water interface. Nanoemulsions may sometimes be created by adding shear force to an existing emulsion.

The use of SEDDS formulation presents multiple advantages as outlined below:
1. By delivering lipophilic and poorly water soluble drugs in a solubilized state using SEDDS, the energy input associated with a solid-liquid phase transition and the slow dissolution process is avoided. This can improve the bioavailability of the drug.
2. In situ phase transition to high viscosity liquid crystalline systems can occur upon dilution with tear fluid. This can increase the formulation residence time on the cornea and improve drug bioavailability.
3. The formation of nano-sized droplets upon dispersion can further improve drug bioavailability due to the potential for direct uptake of nano-sized particles by tissues.
4. Certain surfactant/co-surfactant combinations used in preparing SEDDS can have an enhancing effect on drug permeation across the cornea.
5. The spontaneous self-emulsification gives rise to nanosized emulsions which have a clear appearance due to the small droplet size (e.g., less than 200 nm). Such nanosized emulsions do not cause blurred vision as is commonly experienced with conventional emulsions which is attributed to the larger droplet size and milky-white appearance of the latter. This can help improve patient satisfaction and compliance.
6. In case of SMEDDS which give rise to microemulsion upon dispersion in an aqueous phase, the resultant microemulsions are thermodynamically stable systems and will not breakdown over time.
7. Exclusion of an aqueous component from the final formulation can protect labile API from undergoing hydrolytic degradation and potentially extend the shelf-life of the product.
8. The manufacturing of SEDDS is a simple process with few steps which can be carried out at ambient temperature and does not require a large input of energy. As a result, this can provide enhanced stability for heat sensitive API during manufacturing.

In the SEDDS compositions provided herein, the surfactant is preferably selected from, but not limited to, non-ionic surfactants with HLB>12. “HLB” refers to hydrophilic/lipophilic balance. HLB (Hydrophilic-Lipophilic Balance) is a calculated value to rank non-ionic surfactants with respect to their ability to stabilize emulsions. HLB typically has a scale of 1-20. Surfactants with high HLB values (e.g. >10) are used to stabilize oil-in-water emulsions whereas surfactants with low HLB values (e.g. <8) are used to stabilize water-in-oil emulsions.

Examples of non-ionic surfactants with HLB>12 include, but are not limited to: Polysorbate 80 (polyoxyethylene sorbitan monooleate), Polysorbate 40 (polyoxyethylene sorbitan monopalmitate), Polysorbate 20 (polyoxyethylene sorbitan monolaurate), Cremophor® ELP (purified polyoxyl 35 castor oil), Cremophor® RH-40 (polyoxyl 40 hydrogenated castor oil), Cremophor® A25 (polyoxyl 25 stearyl alcohol ether), Gelucire® 44/14 (lauroyl polyoxyl glycerides), Gelucire® 50/13 (stearoyl polyoxyl glycerides), Labrasol® (caprylocaproyl polyoxyl-8 glycerides), Capryol™ 90 (propylene glycol monocaprylate), Lauroglycol™ 90 (propylene glycol monolaurate), Brij® 97 (polyoxyethylene 10 oleyl ether), and combinations thereof. In some embodiments, the surfactant is selected from the group consisting of Cremophor® ELP, Cremophor® RH-40 or Polysorbate 80 (“PS80”).

In some embodiments, a co-solvent for use in the compositions provided herein may be select from, among others, Transcutol® HP, PEG (polyethylene glycol) 300, PEG 400, propylene glycol, and combinations thereof. In some embodiments, the co-solvent is selected from the group consisting of PEG 400, PEG 300 and propylene glycol.

An oil used in the SEDDs compositions provided herein can be of natural, synthetic, or semi-synthetic origin. The oil may be selected from the group consisting of a single long chain triglyceride, a single medium chain triglyceride, a medium chain monoglyceride, and a medium chain diglyceride. In some embodiments, the oil is a blend of a monoglyceride or a diglyceride blended with either a long chain triglyceride or a medium chain triglyceride.

In some embodiments the SEDDs compositions provided herein are composed of about 5% to about 60% w/w oil. In some embodiments, the compositions contain about 10% to about 40% w/w oil.

In some embodiments, the oil is selected from the group consisting of castor oil, cottonseed oil, soybean oil, olive oil, corn oil, safflower oil, sesame oil, caprylic/capric glyceride (such as Imwitor® 742), glyceryl tricaprylate/tricaprate (such as Captex® 355), propylene glycol dicaprylocaprate (such as Captex® 200P), medium chain mono- and diglycerides (such as Capmul® MCM), caprylic/capric triglycerides (such as Miglyol® 812 and/or Labrafac™ Lipophile WL 1349), glyceryl oleate (such as Peceol™), glyceryl monolinoleate (such as Maisine® 35-1), triacetin, propylene glycol dicaprylate/dicaprate (such as Labrafac™ PG), or combinations thereof. In some embodiments, only one oil is provided in the compositions herein. In some embodiments, the oil is selected from the group consisting of castor oil, Captex®355 and Capmul® MCM. In some embodiments, the oil is castor oil. In some embodiments, the oil is Captex 355. In some embodiments, the oil is Capmul® 355.

In some embodiments, a combination of oils is provided. In some embodiments, the combination of oils is two or more of the following: castor oil, Captex®355 and Capmul® MCM.

In some embodiments, the oil is a mixture of 1:1 by weight of castor oil and Capmul® MCM. In some embodiments, the oil is a mixture of 2:1 by weight of castor oil and Capmul® MCM. In some embodiments, the oil is a mixture of 3:1 by weight of castor oil and Capmul® MCM.

In some embodiments, the oil is a mixture of 1:1 by weight of Capmul® MCM and Captex®355. In some embodiments, the oil is a mixture of 2:1 by weight of Capmul® MCM and Captex®355. In some embodiments, the oil is a mixture of 3:1 by weight of Capmul® MCM and Captex®355.

In some embodiments, the composition comprises castor oil, Cremophor® ELP and PEG 300. In some embodiments, the composition comprises castor oil and 2:1 by weight of Cremophor® ELP:PEG 300.

In some embodiments, the composition comprises castor oil, Capmul® MCM, Cremophor® RH-40, and propylene glycol. In some embodiments, the composition comprises 1:1 by weight of castor oil:Capmul® MCM, and 2:1 by weight of Cremophor® RH-40:propylene glycol.

In some embodiments, the composition comprises Captex®355, PS80, and PEG 400. In some embodiments, the composition comprises Captex®355, and 3:1 by weight of PS80:PEG400.

In some embodiments, the composition comprises Capmul® MCM, Cremophor® RH-40, and propylene glycol. In some embodiments, the composition comprises Capmul® MCM, and 2:1 by weight of Cremophor® RH-40:propylene glycol.

In some embodiments, the composition comprises Capmul® MCM, Cremophor® ELP, and propylene glycol. In some embodiments, the composition comprises Capmul® MCM, and 2:1 by weight of Cremophor® ELP:propylene glycol.

In some embodiments, the composition comprises Capmul® MCM, PS80, and PEG 400. In some embodiments, the composition comprises Capmul® MCM, and 3:1 by weight of PS80:PEG400.

In some embodiments, the composition comprises a oil mixture of 3:1 by weight of castor oil and Capmul® MCM, and a mixture of 3:1 by weight of Cremophor® ELP and PEG 400. In some embodiments, the composition further comprises a 4-pregenen-110-17-21-triol-3,20-dione derivative. In some embodiments, the composition further comprises prednisolone acetate.

In some embodiments, the composition comprises an oil mixture of 2:1 by weight of Captex®355 and Capmul® MCM, and a mixture of 4:1 by weight of Cremophor® ELP and PEG 400. In some embodiments, the composition further comprises a 4-pregenen-110-17-21-triol-3,20-dione derivative. In some embodiments, the composition further comprises prednisolone acetate.

In one embodiment, the composition comprises about 10% to about 40% w/w castor oil, wherein the composition further comprises Cremophor® ELP and PEG 300. In one embodiment, the composition comprises about 10% w/w castor oil, about 60% w/w Cremophor® ELP and about 10% w/w PEG 300. In some embodiments, the composition further comprises a 4-pregenen-110-17-21-triol-3,20-dione derivative. In some embodiments, the composition further comprises prednisolone acetate.

In one embodiment, the composition comprises about 10% to about 40% w/w of a 1:1 mixture of castor oil and Capmul® MCM, wherein the composition further comprises Cremophor® ELP and PEG 300. In one embodiment, the composition comprises about 10% w/w castor oil, about 10% w/w Capmul® MCM, about 53% w/w Cremophor® ELP and about 27% w/w PEG 300. In one embodiment, the composition comprises about 5% w/w castor oil, about 5% w/w Capmul® MCM, about 60% w/w Cremophor® ELP and about 30% w/w PEG 300. In some embodiments, the composition further comprises a 4-pregenen-110-17-21-triol-3,20-dione derivative. In some embodiments, the composition further comprises prednisolone acetate.

In one embodiment, the composition comprises about 10% to about 40% w/w of Captex®355, wherein the composition further comprises PS80 and PEG 400. In one embodiment, the composition comprises about 10% w/w Captex®355, about 67.5% w/w PS80, and about 22.5% w/w PEG 400. In some embodiments, the composition further comprises a 4-pregenen-110-17-21-triol-3,20-dione derivative. In some embodiments, the composition further comprises prednisolone acetate.

In one embodiment, the composition comprises about 10% to about 40% w/w of Capmul® MCM, wherein the composition further comprises Cremophor® RH-40 and propylene glycol. In one embodiment, the composition comprises about 30% w/w Capmul® MCM, about 47% w/w Cremophor® RH-40, and about 24% w/w propylene glycol. In one embodiment, the composition comprises about 20% w/w Capmul® MCM, about 53% w/w Cremophor® RH-40, and about 27% w/w propylene glycol. In one embodiment, the composition comprises about 10% w/w Capmul® MCM, about 60% w/w Cremophor® RH-40, and about 30% w/w propylene glycol. In some embodiments, the composition further comprises a 4-pregenen-11β-17-21-triol-3,20-dione derivative. In some embodiments, the composition further comprises prednisolone acetate.

In one embodiment, the composition comprises about 10% to about 40% w/w of Capmul® MCM, wherein the composition further comprises Cremophor® ELP and propylene glycol. In one embodiment, the composition comprises about 20% w/w Capmul® MCM, about 53% w/w Cremophor® ELP, and about 27% w/w propylene glycol. In one embodiment, the composition comprises about 10% w/w Capmul® MCM, about 60% w/w Cremophor® ELP, and about 30% w/w propylene glycol. In some embodiments, the composition further comprises a 4-pregenen-110-17-21-triol-3,20-dione derivative. In some embodiments, the composition further comprises prednisolone acetate.

In one embodiment, the composition comprises about 10% to about 40% w/w of Capmul® MCM, wherein the composition further comprises PS80 and PEG 400. In one embodiment, the composition comprises about 10% w/w Capmul® MCM, about 67.5% w/w PS80, and about 22.5% w/w PEG 400. In some embodiments, the composition further comprises a 4-pregenen-110-17-21-triol-3,20-dione derivative. In some embodiments, the composition further comprises prednisolone acetate.

In one embodiment, the composition comprises about 10% to about 40% w/w of a 3:1 mixture of castor oil and Capmul® MCM, wherein the composition further comprises Cremophor® ELP and PEG 400. In one embodiment, the composition comprises about 15% w/w castor oil, about 5% w/w Capmul® MCM, about 60% w/w Cremophor® ELP and about 20% w/w PEG 400. In some embodiments, the composition further comprises a 4-pregenen-110-17-21-triol-3,20-dione derivative. In some embodiments, the composition further comprises prednisolone acetate.

In one embodiment, the composition comprises about 10% to about 40% w/w of a 2:1 mixture of Captex®355 and Capmul® MCM, wherein the composition further comprises Cremophor® ELP and PEG 400. In one embodiment, the composition comprises about 27% w/w Captex®355, about 13% w/w Capmul® MCM, about 48% w/w Cremophor® ELP and about 12% w/w PEG 400. In some embodiments, the composition further comprises a 4-pregenen-110-17-21-triol-3,20-dione derivative. In some embodiments, the composition further comprises prednisolone acetate.

Furthermore, a co-surfactant may optionally be used in combination with the surfactants provided herein. In some embodiments, the co-surfactant is non-ionic, with an HLB<10, and is selected from the group consisting of: Span 83, Span 80, Span 60, span 40, Span 20, Capryol™ 90 and Lauroglycol™ 90, or combinations thereof.

In some embodiments, and without being bound by theory or mechanism of action, the non-aqueous SEDDs compositions provided herein do not contain or require a preservative because of the lack of aqueous environment in said compositions. In some embodiments, the non-aqueous SEDDs compositions provided herein do not contain anti-microbial preservatives.

In some embodiments, provided herein is a kit which contains a SEDDs composition provided herein. In some embodiments, the kit is a multi-dose bottle suitable for ophthalmic administration. In some embodiments, the kit is a single-dose vial or container suitable for ophthalmic administration. Such kits are useful for direct application to an eye of a patient in need of treatment of a disease or disorder of the eye.

In some embodiments, the kit comprises two bottles, containers or compartments, one of which contains a non-aqueous SEDDs composition provided herein, and the other of which comprises an ophthalmically acceptable aqueous solution. These two-part systems may be combined by a doctor or patient shortly before administration of the combined solution to the eye of a patient.

### Processes for Preparation

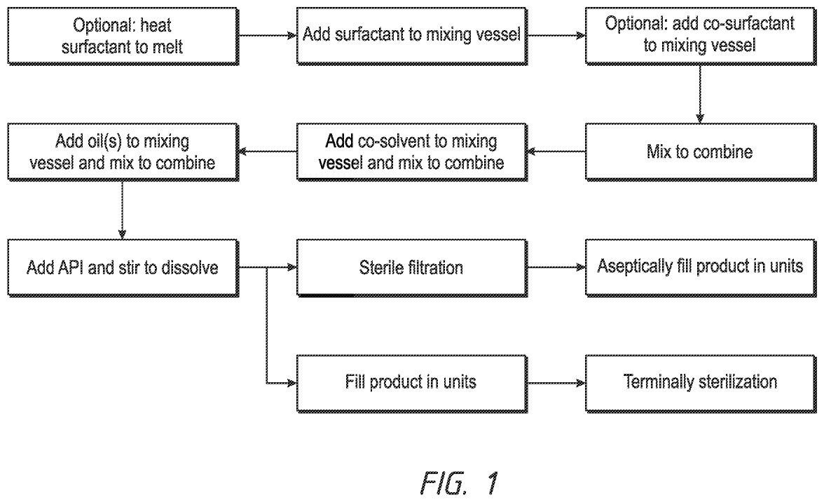

The self-emulsifying systems provided herein can be prepared by the following simple steps (see FIG. 1):
1. Weigh out the appropriate amount of surfactant (for surfactants that are a pasty solid at room temperature, gentle heating is required);
2. If required, add the appropriate amount of co-surfactant and mix to combine;
3. Add the appropriate amount of co-solvent and mix to combine;
4. Add the required amount of oil and mix to combine;
5. Add the pharmaceutically active ingredient and mix to dissolve;
6. Use appropriate sterilization method and product fill.

### EXAMPLES

### Example 1

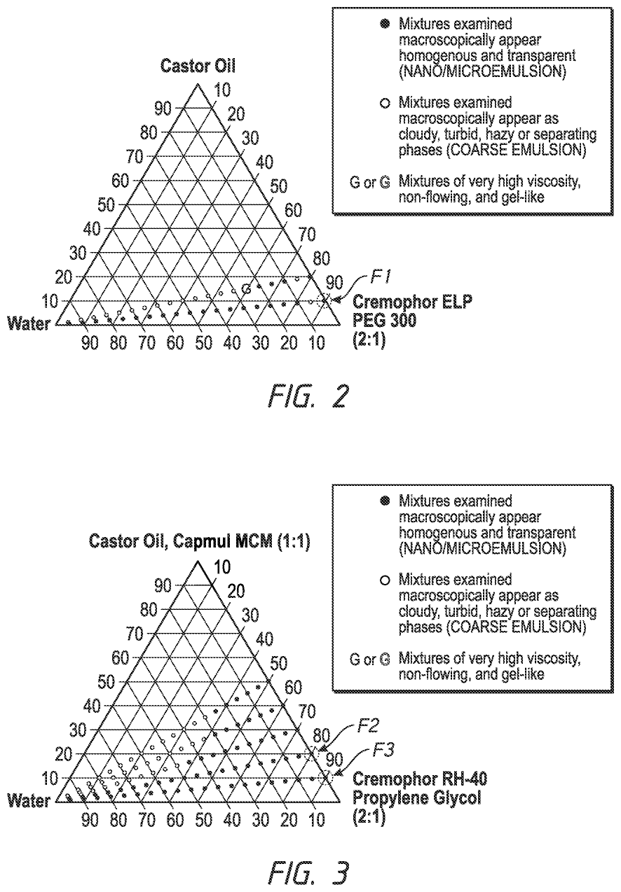

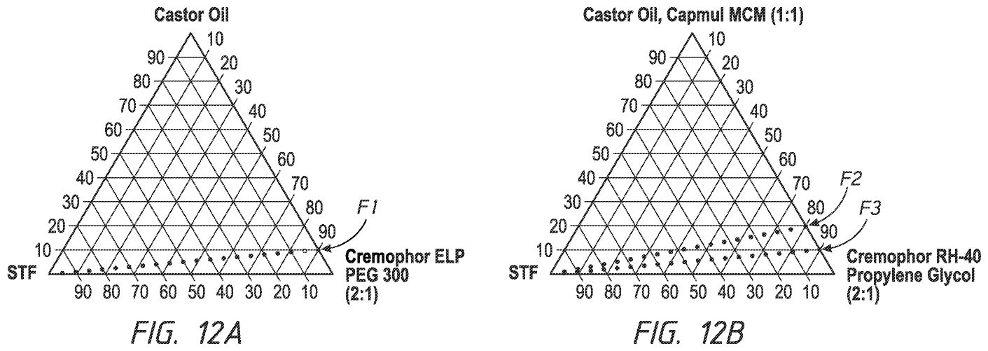

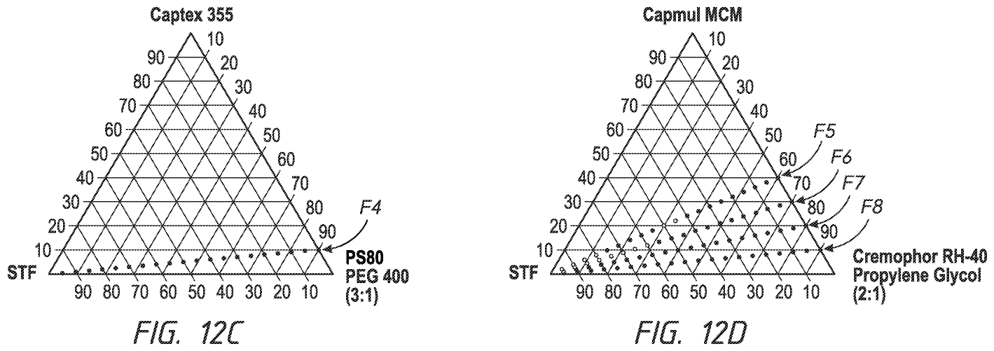

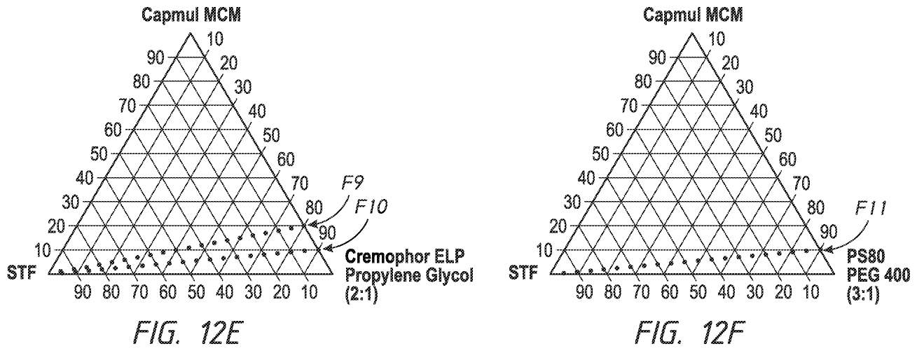

The following example is for a SEDDS formulation where the oil component is a long chain triglyceride from a vegetable source. The ratio of oil to surfactant/co-solvent is varied at either 1:9 or 2:8. The effect of dilution with water up to a final water content of 95% w/w on the appearance of the emulsion can be seen in the phase diagram below in FIG. 2. The surfactant to co-solvent ratio is kept constant at 2:1 so that the effect of increasing oil content on the ability to self-emulsify and generate a clear nanosized emulsion can be isolated. Formulation F1 (Table 2) is selected with a 10% w/w oil content based on the favorable dilution indicated in the phase diagram. Dilution of F1 with simulated tear fluid was subsequently confirmed and showed no impact on nanosized emulsion formation (FIG. 12).

TABLE 2
SEDDS FORMULATION FOR EXAMPLE 1 (F1)

| | Concentration |
|---|---|
| Ingredient | (% w/w) |
| Castor Oil | 10 |
| Cremophor ® ELP | 60 |
| PEG 300 | 30 |

### Example 2

The following example is for SEDDS formulations in which the oil component is a long chain triglyceride blended with a medium chain mono-/di-glyceride in a 1:1 ratio. The inclusion of a medium chain mono-/di-glyceride in the oil component is intended to improve the region of nanosized emulsification as compared to using a long chain triglyceride alone. The surfactant to co-solvent ratio is kept constant at 2:1 and the content of the oil is increased from 10% w/w of formulation up to 50%. Dilution of formulations up to a 95% w/w final water content was performed and the results are illustrated in the phase diagram below in FIG. 3. Formulations F2 and F3 were selected and contain 20% and 10% w/w oil content, respectively. The compositions can be seen in Table 3 and Table 4 below. Dilution with simulated tear fluid was also subsequently confirmed and showed no impact on nanosized emulsion formation (FIG. 12).

TABLE 3
SEDDS FORMULATION FOR EXAMPLE 2 (F2)

| | Concentration |
|---|---|
| Ingredient | (% w/w) |
| Castor Oil | 10 |
| Capmul ® MCM | 10 |
| Cremophor ® ELP | 53.33 |
| PEG 300 | 26.67 |

TABLE 4
SEDDS FORMULATION FOR EXAMPLE 2 (F3)

| | Concentration |
|---|---|
| Ingredient | (% w/w) |
| Castor Oil | 5 |
| Capmul ® MCM | 5 |
| Cremophor ® ELP | 60 |
| PEG 300 | 30 |

### Example 3

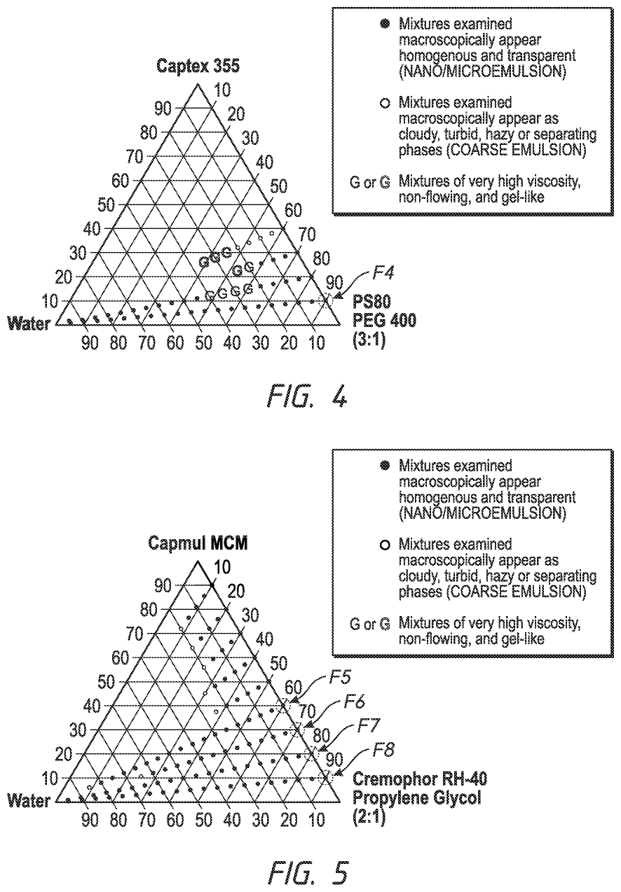

The following example is for a SEDDS formulation containing a medium chain triglyceride, Captex®355, consisting of mixture of caprylic acid (C8) and capric acid (C10) in a 55:45 ratio as the oil component. Formulation F4 is selected from the phase diagram (FIG. 4) on the basis of favorable dilution with water which was further confirmed with simulated tear fluid (FIG. 12). The composition of F4 can be seen in Table 5.

TABLE 5
SEDDS FORMULATION FOR EXAMPLE 3 (F4)

| | Concentration |
|---|---|
| Ingredient | (% w/w) |
| Captex ®355 | 10 |
| PS80 | 67.5 |
| PEG 400 | 22.5 |

### Example 4

The following example is for a system composed of Capmul® MCM as the oil phase and Cremophor® RH-40 and Propylene Glycol as the surfactant and co-solvent, respectively. Capmul® MCM is a synthetic oil of medium chain length mono (60%) and diglyceride (35%) consisting of 83% w/w caprylic acid (C8) and 17% w/w capric acid (C10). Formulations F5, F6, F7 and F8 were selected from the phase diagram (FIG. 5) on the basis of favorable dilution with water which was further confirmed with simulated tear fluid for formulations F7 and F8 (FIG. 12). The composition of these formulations can be seen in Tables 6, 7, 8 and 9.

TABLE 6
SEDDS FORMULATION FOR EXAMPLE 4 (F5)

| | Concentration |
|---|---|
| Ingredient | (% w/w) |
| Capmul ® MCM | 40 |
| Cremophor ® RH-40 | 40 |
| Propylene Glycol | 20 |

TABLE 7
SEDDS FORMULATION FOR EXAMPLE 4 (F6)

| | Concentration |
|---|---|
| Ingredient | (% w/w) |
| Capmul ® MCM | 30 |
| Cremophor ® RH-40 | 46.67 |
| Propylene Glycol | 23.33 |

TABLE 8
SEDDS FORMULATION FOR EXAMPLE 4 (F7)

| | Concentration |
|---|---|
| Ingredient | (% w/w) |
| Capmul ® MCM | 20 |
| Cremophor ® RH-40 | 53.33 |
| Propylene Glycol | 26.67 |

TABLE 9
SEDDS FORMULATION FOR EXAMPLE 4 (F8)

| | Concentration |
|---|---|
| Ingredient | (% w/w) |
| Capmul ® MCM | 10 |
| Cremophor ® RH-40 | 60 |
| Propylene Glycol | 30 |

### Example 5

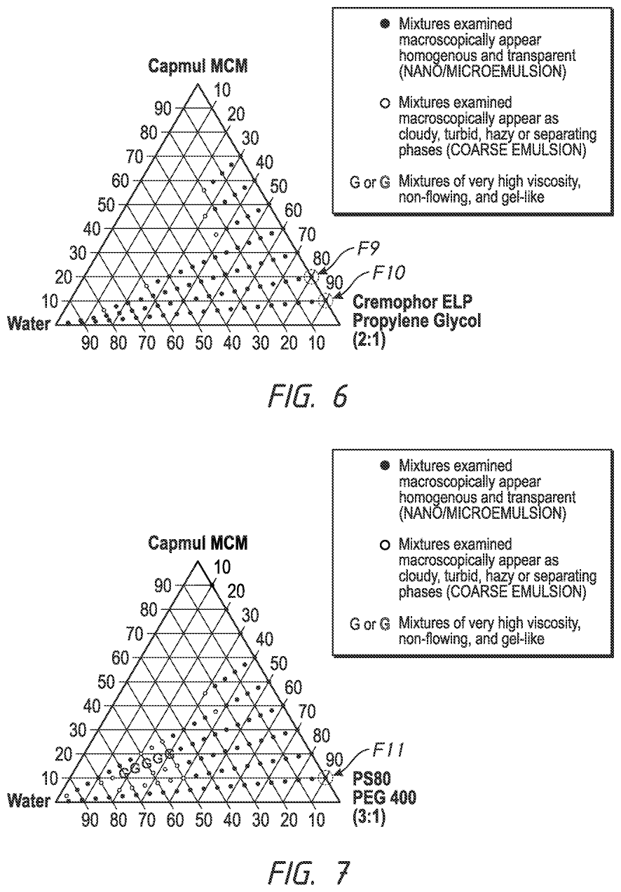

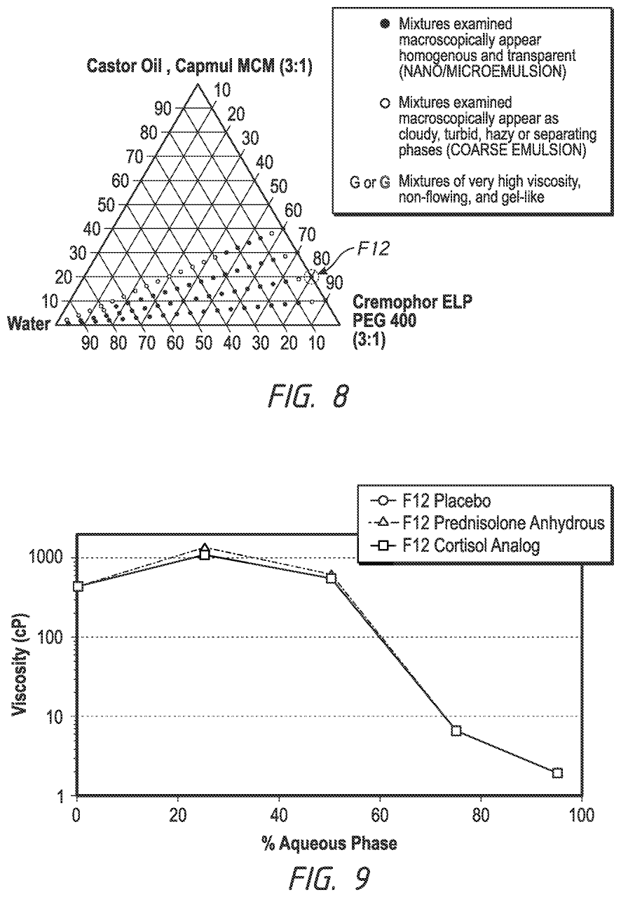

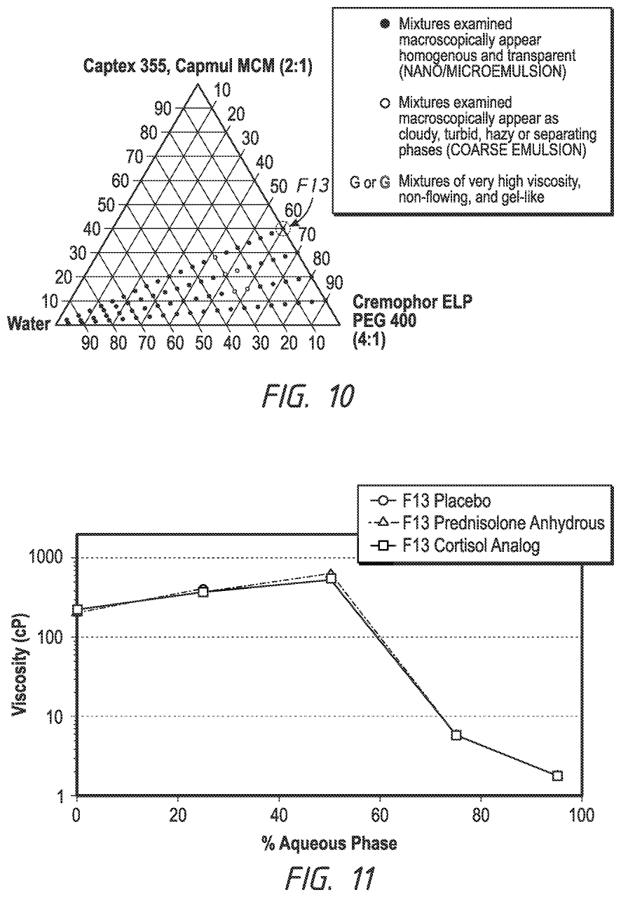

The following example is for a system composed of Capmul® MCM as the oil phase and with Cremophor® ELP and Propylene Glycol as the surfactant and co-solvent, respectively. Formulations F9 and F10 were selected from the pseudo-ternary phase diagram (FIG. 6) and dilution with simulated tear fluid was also later confirmed (FIG. 12). The compositions for these formulations is listed in Table 10 and Table 11 below.

TABLE 10
SEDDS FORMULATION FOR EXAMPLE 5 (F9)

| | Concentration |
|---|---|
| Ingredient | (% w/w) |
| Capmul ® MCM | 20 |
| Cremophor ® ELP | 53.33 |
| Propylene Glycol | 26.67 |

TABLE 11
SEDDS FORMULATION FOR EXAMPLE 5 (F10)

| | Concentration |
|---|---|
| Ingredient | (% w/w) |
| Capmul ® MCM | 10 |
| Cremophor ® ELP | 60 |
| Propylene Glycol | 30 |

### Example 6

In this example, Capmul® MCM was used as the oil phase and PS80 and PEG 400 were used as the surfactant and co-solvent, respectively. Formulation F11 was selected from the following pseudo-ternary phase diagram (FIG. 7) for which the composition is listed in Table 12 below. Again, the compatibility of this formulation with dilution using simulated tear fluid was confirmed (FIG. 12).

TABLE 12
SEDDS FORMULATION FOR EXAMPLE 6 (F11)

| | Concentration |
|---|---|
| Ingredient | (% w/w) |
| Capmul ® MCM | 10 |
| PS80 | 67.5 |
| PEG 400 | 22.5 |

### Example 7

In this example, a blend of Castor oil and Capmul® MCM in a ratio of 3:1 was used as the oil phase. Cremophor® ELP and PEG 400 were used as the surfactant and co-solvent, respectively. Formulation F12 exhibited good dilution with water and was therefore selected. The composition is listed in Table 13. It was noted that this formulation experienced a considerable change in viscosity during aqueous dilution. As such, the change in viscosity upon dilution was measured and a maxima of approximately 1300 cP was observed at 25% aqueous content in the formulation (FIG. 9).

TABLE 13
SEDDS FORMULATION FOR EXAMPLE 7 (F12)

| | Concentration |
|---|---|
| Ingredient | (% w/w) |
| Castor Oil | 15 |
| Capmul ® MCM | 5 |
| Cremophor ® ELP | 60 |
| PEG 400 | 20 |

### Example 8

In this example, a 2:1 blend of two synthetic oils of medium chain length, Captex®355 and Capmul® MCM, were used as the oil phase. Cremophor® ELP and PEG 400 were used as the surfactant and co-solvent, respectively. Formulation F13 exhibited good dilution with water and the composition is listed in Table 14. It was noted that this formulation experienced a change in viscosity during aqueous dilution. As such, the effect of aqueous dilution on the viscosity was measured. A maxima of approximately 600 cP was observed at 50% aqueous content in the formulation (FIG. 11).

TABLE 14
SEDDS FORMULATION FOR EXAMPLE 8 (F13)

| | Concentration |
|---|---|
| Ingredient | (% w/w) |
| Captex ®355 | 26.67 |
| Capmul ® MCM | 13.33 |
| Cremophor ® ELP | 48 |
| PEG 400 | 12 |

The incorporation of a lipophilic drug in the selected formulations (F1-F13) was investigated. Three model drugs were used, prednisolone acetate, prednisolone (anhydrous), and a 4-pregenen-110-17-21-triol-3,20-dione derivative (the “Cortisol Analog”). These compounds were selected on the basis of their poor water solubility and susceptibility to degradation in conventional suspension or solution formulations. Below (Table 15) is the maximum equilibrium solubility of these compounds that could be achieved in formulations F1-F13.

TABLE 15
Drug solubility in formulations

| | Maximum drug solubility (% w/w) | | |
|---|---|---|---|
| | Prednisolone | Prednisolone | Cortisol |
| Formulation | Acetate | Anhydrous | Analog |
| F1 | 0.461 | ND | 1.085 |
| F2 | 0.504 | ND | 1.643 |
| F3 | 0.530 | ND | 1.617 |
| F4 | 0.291 | ND | 0.994 |
| F5 | ND | ND | ND |
| F6 | ND | ND | ND |
| F7 | 0.530 | ND | 1.767 |
| F8 | 0.556 | ND | 1.830 |
| F9 | 0.423 | ND | 1.542 |
| F10 | 0.470 | ND | 1.590 |
| F11 | 0.259 | ND | 0.867 |
| F12 | 0.25 | 0.74 | 1.08 |
| F13 | 0.13 | 0.64 | 0.46 |

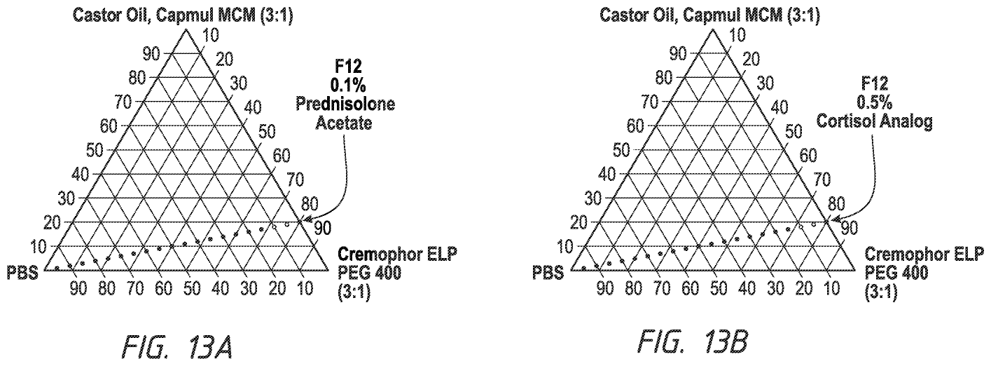

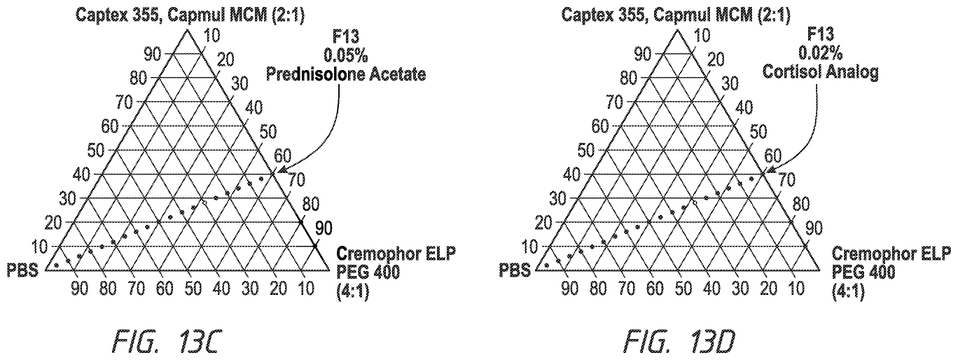

The effect of drug incorporation in the formulation on the ability for self-emulsification upon dilution with an aqueous medium was confirmed. F12 and F13 were selected, and drug loaded formulations were diluted with phosphate buffered saline. The formation of nanosized emulsions upon dilution was unaffected by the presence of drug in both formulations (FIG. 13).

### Ocular Tolerability Study:

The ocular tolerability of the various pharmaceutical-grade excipients used in our formulations was evaluated in vivo using New Zealand White female rabbits. A total of ten test groups with three rabbits each were used to test the materials listed below in Table 16. Dosing in each group was done by instilling one drop of material at the lowest concentration into the left eye of the first rabbit. If not tolerated, dosing was stopped. If tolerated, one drop of the same concentration was instilled into the left eye of the second rabbit and then again for the third rabbit. If a concentration was tolerated by 3 rabbits, dosing continued in the same pattern with the next higher concentration, if applicable.

The maximum tolerated doses and the reason for a “not tolerated” observation are listed in Table 16 below. Sample compositions, specifically, the vehicle used to dilute the materials tested, are listed in the next table (Table 17).

TABLE 16
Ocular tolerability results for materials tested in vivo in New Zealand White rabbits

| | | Maximum | |
|---|---|---|---|
| | | Tolerated | |
| | Concentration(s) to be | Concentration | Reason “Not Tolerated” (if |
| Material Tested | tested (% w/w) | (% w/w) | applicable) |
| Castor Oil | 100 | 100 | N/A |
| Captex ®355 | 100 | 100 | N/A |
| Capmul ® MCM | 100 | N/A | +2 Conjunctival swelling and |
| | | | severe tearing noted 1 hour |
| | | | post dose |
| Capmul ® MCM | 5, 10, 15, 20, 25 | N/A | +3 Ocular discomfort, and +1 |
| | | | Conjuctival swelling seen at |
| | | | 5% |
| Cremophor ® | 10, 20, 30, 40, 50, 60 | 60 | N/A |
| ELP | | | |
| Cremophor ® | 10, 20, 30, 40, 50, 60 | 30 | +3, 3 Ocular discomfort seen at |
| RH-40 | | | 40% |
| PS80 | 10, 20, 30, 40, 50, 60, | 30 | +3, 2 Ocular discomfort seen at |
| | 70 | | 40% |
| PEG 300 | 10, 20, 30, 40 | N/A | +3, 2 Ocular discomfort seen at |
| | | | 10% |
| PEG 400 | 10, 20, 30, 40 | 10 | +3, 1 Ocular discomfort seen at |
| | | | 20% |
| Propylene | 10, 20, 30, 40 | N/A | +3, 1 Ocular discomfort seen at |
| Glycol | | | 10% |

TABLE 17
Sample description for tested materials

| | Concentrations prepared (% | |
|---|---|---|
| Material Tested | w/w) | Diluent used (if applicable) |
| Castor Oil | 100 | N/A |
| Captex ®355 | 100 | N/A |
| Capmul ® MCM | 100 | N/A |
| Capmul ® MCM | 5, 10, 15, 20, 25 | Phosphate Buffered Saline and Cremophor ® |
| | | ELP* |
| Cremophor ® ELP | 10, 20, 30, 40, 50, 60 | Phosphate Buffered Saline |
| Cremophor ® RH-40 | 10, 20, 30, 40, 50, 60 | Phosphate Buffered Saline |
| PS80 | 10, 20, 30, 40, 50, 60, 70 | Castor Oil |
| PEG 300 | 10, 20, 30, 40 | Phosphate Buffered Saline |
| PEG 400 | 10, 20, 30, 40 | Phosphate Buffered Saline |
| Propylene Glycol | 10, 20, 30, 40 | Phosphate Buffered Saline |

*Cremophor ® ELP concentration < 60% w/w

Of the three oils tested, Castor oil and Captex®355 were well tolerated at 100%, while Capmul® MCM was not tolerated at 100%. Of the three surfactants tested, Cremophor® ELP was the most well tolerated (tolerated up to the maximum tested concentration of 60%) while Cremophor® RH-40 and PS80 were both tolerated up to 30%. PEG 400 was the only co-solvent tolerated at 10%, while PEG 300 and Propylene Glycol were not tolerated at 10%. Moderate (+3) discomfort was observed at the lowest tested concentrations (10%) of PEG 300 and Propylene Glycol and mild (+2) conjunctival swelling was observed with 100% Capmul® MCM.

The terms “a,” “an,” “the” and similar referents used herein (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A non-aqueous topical ophthalmic composition comprising an oil, a poorly water-soluble drug, and one or more surfactants;
wherein the composition is capable of self-emulsifying upon mixture with an aqueous solution upon instillation to the eye; and
wherein about 5% to about 60% w/w of the composition consists of the oil.

2. The composition of claim 1, wherein the composition contains less than 1% by weight water.

3. The composition of claim 1, wherein the composition further comprises one or more co-solvents.

4. The composition of claim 1, wherein upon reconstitution in an aqueous medium, the composition forms a stable oil-in-water nanosized emulsion.

5. The composition of claim 3, wherein the nanosized emulsion comprises dispersed oil droplets after self-emulsification are in the size range of 10-200 nm.

6. The composition of claim 1, wherein the oil is selected from the group consisting of a single long chain triglyceride, a single medium chain triglyceride, a medium chain monoglyceride, and a medium chain diglyceride.

7. The composition of claim 1, wherein the oil is a blend of a monoglyceride or a diglyceride blended with either a long chain triglyceride or a medium chain triglyceride.

8. The composition of claim 1, wherein the oil is castor oil.

9. The composition of claim 1, wherein the oil is Captex®355 or Capmul® MCM, or a combination thereof.

10. The composition of claim 8, wherein the surfactant is selected from the group consisting of Cremophor® ELP, Cremophor® RH-40, and polysorbate 80.

11. The composition of claim 3, wherein the co-solvent is selected from the group consisting of polyethylene glycol 300, polyethylene glycol 400, and propylene glycol.

12. The composition of claim 1, wherein the composition comprises about 10% to about 40% w/w oil.

13. The composition of claim 12, wherein the oil is selected from the group consisting of castor oil, Captex®355, Capmul® MCM and mixtures thereof.

14. The composition of claim 12, wherein the oil is castor oil.

15. The composition of claim 14, wherein the composition further comprises Cremophor® ELP and polyethylene glycol 300.

16. The composition of claim 15, wherein the composition comprises about 10% w/w castor oil, about 60% w/w Cremophor® ELP and about 10% w/w polyethylene glycol 300.

17. The composition of claim 13, wherein the oil is a 1:1 w/w mixture of castor oil and Capmul® MCM.

18. The composition of claim 17, wherein the composition further comprises Cremophor® ELP and polyethylene glycol 300.

19. The composition of claim 18, wherein the composition comprises about 10% w/w castor oil, about 10% w/w Capmul® MCM, about 53% w/w Cremophor® ELP and about 27% w/w polyethylene glycol 300.

20. The composition of claim 18, wherein the composition comprises about 5% w/w castor oil, about 5% w/w Capmul® MCM, about 60% w/w Cremophor® ELP and about 30% w/w polyethylene glycol 300.

21-46. (canceled)
